(19) United States
(12) Patent Application Publication
(10) Pub. No.: US 2009/0209633 A1
(43) Pub. Date: Aug. 20, 2009

(54) Treatment of Pain With Resiniferatoxin and Related Analogs

(76) Inventor: Louis S. Premkumar, Springfield, IL (US)

Correspondence Address: SIU SCHOOL OF MEDICINE;OFFICE OF TECHNOLOGY TRANSFER, P.O. BOX 19616, SPRINGFIELD, IL, 62794, US

(21) Appl. No.: 12/356,469

(22) Filed: Jan. 20, 2009

Related U.S. Application Data
(63) Continuation-in-part of application No. PCT/US07/73913, filed on Jul. 19, 2007.
(60) Provisional application No. 60/807,905, filed on Jul. 20, 2006.

(51) Int. Cl. A61K 31/357; C12N 5/06; A61P 25/00

(52) U.S. Cl. 514/450; 435/375

(57) ABSTRACT

A method of treating inflammatory pain conditions is provided that involves administering an effective amount of a TRPV1 agonist, such as resiniferatoxin, tinyatoxin and related potent agonists and their analogs, to a patient to selectively induce nerve terminal depolarization block and/or nerve terminal death in select TRPV1-containing neurons, to provide the desired pain relief without significant permanent damage to cell bodies of the select TRPV-1 containing neurons.

### CROSS REFERENCE TO RELATED APPLICATIONS

This application is a continuation-in-part of International Application No. PCT/US2007/073913, filed Jul. 19, 2007, claiming the benefit of U.S. Provisional Patent Application No. 60/807,905, filed Jul. 20, 2006, both of which are incorporated by reference herein in their entirety.

### FIELD OF THE INVENTION

The present invention relates to treatment of chronic pain conditions including chronic inflammatory pain and neuropathic conditions. More specifically, this invention relates to methods of treating chronic inflammatory pain and neuropathic conditions using TRPV1 agonists, such as resiniferatoxin (RTX), tinyatoxin, capsaicin, iodoRTX (an antagonist that acts as an agonist upon dissociation of iodine) and related potent agonists and their analogs. Although not wishing to be limited by theory, it is believed that these methods reduce nociceptive transmission by inducing selective nerve terminal depolarization block in the short term leading to nerve terminal death in the long term in the central and/or peripheral terminals of select TRPV1-containing neurons, without significant permanent damage to the cell bodies of the select TRPV1-containing neurons.

### BACKGROUND

Chronic pain is a costly health problem in America. Pain is a frequent cause for clinical visits, with approximately 45% of the population seeking medical help for pain at some point in their lives. (Statement by NIH Dec. 8, 2005.) Pain occurs across the life span, yet a significant portion of people with moderate to severe pain do not get adequate relief. Low back pain, in particular, is a significant health problem affecting the majority of people at some point their life. Other significant sources of chronic pain include cancer pain, arthritic pain, muscle pain, burn pain, headache, inflammatory bowel conditions, visceral pain, and other pain disorders such as the neuralgias and neuropathies that affect nerves throughout the body, pain due to damage to the central nervous system (the brain and spinal cord), as well as pain where no physical cause can be found and/or psychogenic pain.

One estimate suggests that nearly 20% of the world's population suffer from some type of chronic pain, and it is recognized that pain relief should be a fundamental right of those suffering. (Medical News Today Aug. 25, 2005.) In Europe alone, it is estimated that nearly 500 million working days are lost each year as a result of chronic pain. (Id.) It is also estimated that over 86 million American adults suffer from some degree of chronic pain. (Pain Management Health Center Jun. 5, 2006.) Chronic pain is a disorder that can persist for months or years and which cannot be fully relieved with standard pain medications. Chronic pain is widely believed to represent a disease itself, causing long-term detrimental changes in the nervous system. It manifests, however, with many other physiologic and psychosocial disorders, including depression and anxiety, increasing the disability and impairment of these conditions. Pain may interfere with sleep, activities of daily living, and productivity. It lowers the quality of life and is a risk factor for suicide in patients suffering from depression. Chronic pain is an enormous burden on health care resources.

Current treatments for severe chronic pain are limited in their application and scope. They generally require a patient to undergo a complete numbing process that impedes all sensation in a selected area or the use of pain medications such as prescription strength narcotics. These treatments result in an undesirable loss of normal nervous system functioning (often in areas of the body distant from the pain source), respiratory depression and other adverse side effects, and/or increased risk of drug dependency. Thus, it would be desirable to provide alternative methods of treating chronic pain without, or with reduced risk of, these undesirable effects.

Transient receptor potential vanilloid 1 (TRPV1 or VR1) is a non-selective cation channel with high calcium permeability expressed on the peripheral and central terminals of small-diameter sensory neurons. (Caterina et al. 1997; Dinh et al. 2004; Lazzeri et al. 2004c.) It functions as a polymodal nociceptor at the peripheral nerve terminals and modulates synaptic transmission at the first sensory synapse between dorsal root ganglion (DRG) and dorsal horn neurons. (Nakatsuka et al. 2002; Baccei et al. 2003.) TRPV1 has also been shown to modulate synaptic transmission in certain regions of the brain. (Doyle et al. 2002; Marinelli et al. 2002, 2003.) TRPV1 is activated by heat (>42° C.), capsaicin (pungent ingredient of hot chilli peppers), resiniferatoxin (RTX), protons, anandamide, arachidonic acid metabolites and N-arachidonyl dopamine (NADA). (Szallasi et al. 1990a,b; Caterina et al. 1997; Zygmunt et al. 1999; Hwang et al. 2000; Julius et al. 2001; Caterina et al. 2001; Chuang et al. 2001; De Petrocellis et al. 2001; Huang et al. 2002.) RTX, derived from Euphorbia resinifera, is the most potent amongst all the known endogenous and synthetic agonists for TRPV1. The tritiated form (³[H]RTX) has been used as a tool in ligand-binding assays. (Szallasi et al. 1990b; Roberts et al. 2004.)

Binding of capsaicin and RTX to TRPV1 involves amino acid residues which have been shown to reside in N- and C-cytosolic and transmembrane domains of the channel. (Jung et al. 1999, 2002; Chou et al. 2004; Gavva et al. 2004.) RTX, the structure of which is shown below, combines structural features of phorbol esters (potent activators of protein kinase C (PKC)) and vanilloid compounds. It was thought that its ability to activate PKC might be responsible for its high potency, but the concentration required to activate PKC is much higher than needed to account for this effect. (Harvey et al. 1995.)

### RTX

TRPV1 is implicated in inflammatory thermal sensitivity, as TRPV1 knockout mice are able to sense normal temperature with some deficiency, but lack thermal hypersensitivity following inflammation. (Caterina et aL 2000; Davis et al. 2000.) Although TRPV1 is mainly considered to be involved in thermal sensory perception, its distribution in regions that are not exposed to such temperatures raises the possibility of functions other than detection of heat. TRPV1 can be detected using RT-PCR and radioligand binding throughout the neuroaxis, and identification of specific ligands such as NADA in certain brain regions further suggests possible roles in the CNS. (Huang et al. 2002; Szabo et al. 2002; Zheng et al. 2003; Vass et al. 2004.) TRPV1 is present in the blood vessels and bronchi where activation of this receptor leads to potent vasodilatation (by releasing calcitonin gene-related peptide (CGRP)) and bronchoconstriction, respectively. (Lundberg et al. 1983; Mitchell et al. 1997; Oroszi et al. 1999.) TRPV1 is found in the nerve terminals supplying the bladder and the urothelium, where activation may have a role in bladder function, including micturition. (Birder et al. 2002; Linard et al. 2003; Dinis et al. 2004.)

Recently, RTX has found therapeutic usefulness and is undergoing clinical trials for the treatment of bladder hyper-reflexia. (Lazzeri et al. 1998; Kim et al. 2003.) Single intravesicular administration of RTX produces a long-lasting improvement of this condition. (Cruz et al. 1997; Lazzeri et al. 1998; Brady et al. 2004; Karai et al. 2004.) It has also been found that RTX is useful in painful conditions affecting joints where its injection into the joint cavity has led to a dramatic improvement in joint mobility by reducing pain. (Helyes et al. 2004.)

The rationale for RTX treatment is believed to arise from a combination of Ca²⁺-dependent desensitization and the nerve terminals undergoing cell death from excessive influx of Ca²⁺ via TRPV1. The long-lasting effect of RTX supports the latter as a more likely mechanism of action as shown by the effect of RTX administration into the bladder of patients with bladder hyper-reflexia. (Brady et al. 2004.) It has been documented that intravesicular application of RTX, unlike capsaicin, does not induce suprapubic discomfort. (Giannantoni et al. 2004.)

It has been proposed to use RTX for deletion of specific heat-pain-sensing neurons such as C-fibers (which express large amounts of TRPV1 on their surface) for treating chronic pain symptoms in animals. (NIDCR May 3, 2004.) The technique involves injection of RTX into the trigeminal ganglion, or the cerebrospinal fluid that bathes the dorsal root ganglia (DRG). The RTX-induced flow of calcium into the C-fibers can disable and ultimately kill these neurons. Thus, the technique selectively deletes certain neurons but leaves others untouched. As a result, a certain spectrum of pain responsiveness is deleted, but the nervous system otherwise functions essentially normally.

### SUMMARY OF THE INVENTION

The present invention permits the control of chronic inflammatory pain and neuropathic conditions while allowing the nerve cells to regenerate and, therefore, allowing the patient to maintain the ability to regain lost sensations. We have determined that even at low concentrations (i.e., lower than generally used in the prior art), RTX (an ultrapotent agonist) is able to activate TRPV1 slowly with high potency, which might result in a sustained increase in intracellular Ca²⁺ without generating action potentials, leading to nerve terminal death. Thus, this refined method provides an effective method to treat chronic pain conditions including inflammatory pain conditions, which is highly selective, targeting only the terminals of certain neurons, and which also desirably prevents permanent damage to the nerve cell body, thereby providing the patient the ability to regain lost sensations.

According to one aspect of the invention, a novel method of treating inflammatory pain conditions is provided that involves administering an effective amount (generally, the lowest amount that is effective for pain relief) of a TRPV1 agonist, such as RTX, tinyatoxin, or related potent agonists and their analogs, to a patient to selectively induce nerve terminal depolarization block and/or nerve terminal death in select TRPV1-containing neurons without permanently damaging cell bodies in the select TRPV1-containing neurons.

For purposes of this invention, “effective amount” and/or “low concentrations” of the TRPV1 agonist is intended to mean an amount sufficient to provide the desired pain relief by selectively inducing nerve terminal depolarization block and/or nerve terminal death in select TRPV1-containing neurons without permanently damaging a significant proportion of cell bodies in the select TRPV1-containing neurons, thereby allowing the nerve terminals, over time, to regenerate to obtain essentially normal nerve function in the treatment area. Regeneration of nerve terminals is expected in about 4-6 weeks. (See Roberts et al 2004.)

Preferably, the dosage rate is optimized such that lowest concentration effective to provide the desired pain relief is used so as not to cause permanent damage to a significant proportion of cell bodies. Higher concentrations may be used, particularly in cases of extreme pain caused by a terminal condition (for example, bone cancer), to provide the desired pain relief with an acceptably small amount of permanent damage to the nerve cell bodies. Preferably, at least 80% of the nerve cell bodies will remain intact. More preferably, at least 90% of the nerve cell bodies will remain intact, and most preferably, essentially all of the nerve cell bodies will remain intact.

Thus, in one preferred form, the method of the present invention comprises contacting low concentrations of RTX, an ultrapotent TRPV1 agonist, to select TRPV1-containing neurons to reduce nociceptive transmission by inducing selective nerve terminal depolarization block in the short term and nerve terminal death in the long term without permanently damaging cell bodies in the select TRPV1-containing neurons.

According to one aspect of the invention, low concentrations of RTX, preferably in the range of about 0.01 micrograms/kg to about 5 micrograms/kg, and more preferably in the range of about 0.01 micrograms/kg to about 0.5 micrograms/kg, may be introduced into a patient by intrathecal administration, for example, for treatment of chronic inflammatory and neuropathic pain conditions such as cancer pain (particularly bone cancer) and visceral pain, or by intraarticular administration, for treatment of arthritic pain, for example. Preferably, the RTX is provided in a volume of about 0.01 to about 0.1 milliliters of a pharmaceutically acceptable carrier. Alternatively, low concentrations of RTX, preferably about a 5-50 nM solution, may be applied topically, for treatment of burn pain or pain induced by Herpes zoster or AIDS, for example. Thus, methods of application will vary due to the pain to be treated; based on guidance provided herein, one of ordinary skill in the art can determine the best mode of application in a particular case.

The method of the present invention permits treatment of chronic inflammatory pain conditions with a long duration of action. In the preferred form, nerve terminals, especially the central terminals, are selectively targeted by intrathecal administration of RTX. This method allows the DRG neuronal cell body to remain intact and facilitates the regrowth of nerve terminals, thus avoiding permanent damage to TRPV1-containing neurons, especially the cell bodies. Thus, the present method provides an advantage over current methods in which neuronal cell bodies are often permanently damaged thereby preventing significant regeneration of nerve terminals.

### BRIEF DESCRIPTION OF THE DRAWINGS

FIG. 1 shows RTX-induced cloned TRPV1 currents;

FIG. 2 shows TRPV1-mediated whole-cell currents in DRG neurons;

FIG. 3 shows single-channel current recordings in cell-attached patches from DRG neurons;

FIG. 4 shows single-channel current recordings in an excised patch from oocytes injected with cRNA for TRPV1;

FIG. 5 shows multiple conductance states of RTX-induced TRPV1 currents;

FIG. 6 shows voltage dependence of RTX-induced single channel activity in cell-attached patches from DRG neurons;

FIG. 7 shows voltage dependence of capsaicin-induced single channel activity in cell attached patches from DRG neurons;

FIGS. 8A and B are tables showing single-channel kinetics of TRPV1 currents induced by RTX and capsaicin respectively;

FIG. 9 shows RTX- and capsaicin-induced membrane depolarization and the ability to generate action potentials;

FIG. 10 shows RTX-induced nocifensive behavior;

FIG. 11 shows TRPV1-mediated synaptic transmission;

FIG. 12 shows photographs demonstrating the reduction of TRPV1 levels in spinal cord dorsal horn (DH) after intrathecal RTX administration indicating that selective ablation of the nerve terminals occurred after intrathecal RTX administration;

FIG. 13 shows photographs demonstrating the maintenance of TRPV1 levels in DRG after intrathecal RTX administration indicating that the DRG nerve cell bodies remained intact after intrathecal RTX administration;

FIG. 14 shows photographs of TRPV1 levels in paw skin after intrathecal RTX administration; and

FIG. 15 shows nocifensive behavior after intrathecal RTX administration indicating that paw withdrawal latency to radiant heat was not affected but that nocifensive behavior, as indicated by the number and duration of guarding, was significantly reduced after intrathecal administration of RTX.

### DETAILED DESCRIPTION

The present invention provides a method for treating chronic inflammatory pain and neuropathic conditions involving introduction of TRPV1 agonists into a patient to reduce nociceptive transmission by selectively targeting TRPV1 containing nerve terminals to induce selective nerve terminal depolarization block and, ultimately, nerve terminal death. Importantly, the present method does not induce significant permanent damage to the nerve cell body of the nerve itself, thereby allowing regeneration of nerve terminals.

We have determined the characteristics of RTX-induced responses in cells expressing native and cloned rat TRPV1. (See, for example, Raisinghani et al. 2005, the entire disclosure of which is hereby incorporated by reference.) Specifically, our research has confirmed that RTX is a potent agonist of TRPV1 and that the whole-cell current response and single-channel current activity cannot be reversed readily, consistent with its high affinity for the receptor. (Szallasi et al. 1990b; Caterina et al. 1997.) This property of RTX has proven to be a useful tool to study interactions of agonists and antagonists with TRPV1 in receptor-binding experiments using its tritiated form. It is interesting that despite being an ultrapotent agonist with very high affinity, several weaker agonists can displace RTX from its binding site over time. Several studies have identified residues in the extracellular and intracellular domains that are involved in RTX binding. (Jung et al. 1999, 2002; Chou et al. 2004; Gavva et al. 2004.) The dichotomy between affinity and functional effects shown by some studies raises interesting questions about the functional relevance of residues identified as critical for RTX and capsaicin binding. (Gavva et al. 2004.) It also underscores the importance and relevance of the action of RTX on TRPV1, especially in light of its usefulness for certain painful conditions, including urinary bladder hyper-reflexia. (Helyes et al. 2004; Giannantoni et al. 2004; Palma et al. 2004.)

Although RTX is a potent agonist, we have found that the rate of activation of TRPV1 current is slower than with capsaicin and protons. Binding sites for capsaicin and RTX have been identified in the cytosolic and transmembrane domains of the channel, and the lipophilicity of the agonist affecting the ability of the drug to cross the membrane and interact with its respective binding site(s) might contribute to the differences observed in the activation kinetics. (Jung et al. 1999, 2002; Chou et al. 2004; Vyklicky et al. 2003.) Low-potency agonists, such as olvanil and capsiate, have been shown to exhibit low pungency, which is attributed partly to their high lipophilicity. Subcutaneous administration of these low-potency agonists induced nocifensive behavior; however, no effect was seen when they were applied on the skin or mucous membranes. (lida et al. 2003; Neubert et al. 2003.) This discrepancy might be in part due to low levels of the agonists being able to access the nerve terminals when applied superficially. In some in vivo studies, RTX induced an increase in the threshold for paw withdrawal latency in the hot plate test (Szabo et al. 1999; Almasi et al. 2003); however, nocifensive behavior was observed in other studies (e.g., eye wipe tests). (Szallasi et al. 1989.) Furthermore, intravesicular administration of RTX did not induce suprapubic discomfort, unlike that seen following capsaicin administration (Giannantoni et al. 2004).

These findings are consistent with the observation that the ability of RTX to induce action potentials follows a bell-shaped curve, in that there was a significant increase in the number of action potentials only at intermediate concentrations. The property of slow activation may be relevant as the receptor potential is changed in a ramp-like fashion, which is not rapid enough to induce a concerted activation of voltage-gated Na⁺ channels to generate an action potential at the nerve terminal. This property might contribute to the lack of painful burning sensation with RTX, which is observed with capsaicin.

Moreover, the RTX-induced response is long lasting; therefore much lower concentrations of the drug than are being used currently (50-100 nM) could potentially be used to achieve maximal activation of the receptor over a period of time in a therapeutically safe manner while avoiding potential side effects due to systemic absorption. (Giannantoni et al. 2004; Palma et al. 2004.) Current clinical trials for bladder hyper-reflexia are being conducted to determine the most effective concentration of RTX and the duration of its application for optimum clinical benefits. (Lazzeri et al. 2004a,b; Payne et al. 2005.) Vanilloid agonists exhibit different potencies for receptor binding and Ca²⁺ uptake assays. (Acs et al. 1995; Walpole et al. 1996.) RTX was found to be 25-fold more potent for binding (K_{d}, 40 μM) as compared to its ability to induce Ca²⁺ uptake (K_{d}, 1.0 nM). (Acs et al. 1995.) An EC₅₀ of 270 nM was determined for capsaicin when Ca²⁺ influx was used as a parameter. However, [³H]RTX binding was inhibited by capsaicin with a 10-fold lower affinity (K_{d}, 3 μM). (Acs et al. 1995.) It was postulated that two different types of vanilloid receptors, denoted as R-type and C-type vanilloid receptors, could be mediating these distinct responses. (Szallasi et al. 1996; Biro et al. 1997.) However, both R- and C-type responses could be seen in cell lines that expressed cloned vanilloid receptor TRPV1, ruling out the possibility of two different receptor types.

While determining the potency of RTX and capsaicin to activate TRPV1 current, it was found that RTX was 20-fold more potent (EC₅₀, 39 nM) than capsaicin (EC₅₀, 710 nM). (Caterina et al. 1997.) In TRPV1 transfected human embryonic kidney (HEK293) cells, capsaicin activated a current with an EC₅₀ of 110 nM. (Tominaga et al. 1998.) We have found that a meaningful dose-response curve can not be constructed because repeated application of submaximal concentration of RTX induced larger currents until a maximal response was attained. Although RTX is a potent agonist, the activation of the current is slower than with capsaicin, and it deactivates minimally. The high potency is indicated by the minimal deactivation of the whole-cell currents, which is a result of its high affinity for the channel. The presence of critical intracellular residues, which need to be accessed by passing through the membrane, might be one of the contributing factors for the slow activation kinetics of RTX-induced membrane currents. It is also possible that RTX has both agonistic and antagonistic actions and therefore acts as a partial agonist. This is conceivable given the finding that iodoRTX is a potent antagonist of TRPV1. To better understand the nature of activation of TRPV1 by RTX, we have characterized the single-channel properties of RTX- and capsaicin-induced channel activity.

We have used cell-attached and excised patch configurations to record single-channel currents activated by RTX and compared the properties with single-channel currents activated by capsaicin. A clear dose-dependent increase in Pₒ was not observed with RTX; only the time to reach the maximal Pₒ decreased with increasing concentrations. A concentration of 0.1-0.2 nM could maximally activate the receptor. Furthermore, there was no voltage-dependent change in Pₒ with RTX, whereas capsaicin-induced current shows a steep voltage-dependent decrease in Pₒ at negative potentials, which is consistent with earlier work. (Premkumar et al. 2002; Voets et al. 2004.) It is possible that binding of capsaicin is voltage dependent, which is reflected as a reduction in Pₒ. Despite maximal activation of the receptor by RTX, single-channel conductance shows clear outward rectification. The lesser extent of RTX-induced whole-cell current rectification could be due to a lack of voltage-dependent reduction in Pₒ at negative membrane potentials. As maximal activation of TRPV1 by RTX occurs even at low concentrations, it provides a means of studying the gating properties without interference from binding events, in that the equilibrium is greatly favored towards the open state. Therefore, we were able to separate the gating events from binding events to study kinetics in detail. Open-time distributions show that the time constants are longer and that their areas of distribution are greater in the presence of RTX than in the presence of capsaicin. The channel predominantly dwelled in the longer open states as reflected by the fractional area of distribution. Also, consistent with the high mean open times and Pₒ, the closed-time distribution could be fitted in most patches with only two exponential components, and even if a third exponential component was required, the area of its distribution was negligible.

It is clear that the concentrations of RTX and capsaicin that brought the membrane potentials rapidly to threshold, generated action potentials. With lower concentrations of capsaicin, the membrane potential did not even reach the threshold. On the other hand, very low concentrations of RTX, given its irreversible nature, induced a sustained depolarization and changed the membrane potential in a ramp-like fashion beyond the threshold, and yet it either failed to generate, or generated few action potentials in some cells. Thus, there are two advantages of using RTX. First, because of its irreversible nature, very low concentrations (well below the toxic levels) could be applied locally. Second, slow and sustained depolarization results in a gradual inactivation of voltage-gated Na⁺ channels causing a depolarization block, which prevents action potential generation. However, at the same time RTX can sufficiently increase intracellular Ca²⁺ levels via TRPV1 to induce nerve terminal death for pain relief. As the nerve terminals have the ability to regenerate, long-term toxicity may not be a major concern.

It is important to understand the properties of RTX-induced TRPV1 currents because of its potential usefulness as a therapeutic agent. RTX is currently undergoing clinical trials, and showing beneficial effects in rodent models, for the treatment of bladder hyper-reflexia. A single intravesicular or intra-articular administration of RTX produces a long-lasting amelioration of the condition. (Brady et al. 2004; Helyes et al. 2004.) Its long-term beneficial effects are induced by sustained activation of TRPV1, potentially increasing intracellular Ca²⁺ levels, which subsequently induces nerve terminal death. (Cruz et al. 1997; Lazzeri et al. 1998; Giannantoni et al. 2004; Brady et al. 2004; Karai et al. 2004; Lazzeri et al. 2004b,c.) Slow and sustained activation at lower concentrations might contribute to the lack of pungency of RTX, as compared to capsaicin, which favors its usefulness during intravesicular administration.

Thus, according to one aspect of the invention, a method of treating inflammatory pain conditions is provided in which low concentrations of TRPV1 agonists are introduced into a patient to reduce nociceptive transmission by inducing selective nerve terminal depolarization block and, ultimately, nerve terminal death in select TRPV1-containing neurons, without permanently damaging cell bodies of the select TRPV1-containing neurons.

RTX is the preferred TRPV1 agonist for use in the method of the present invention because of its unique properties described above, in particular, its selectivity of action and the very low concentrations required for effectiveness. However, other suitable TRPV1 agonists include, for example, tinyatoxin, capsaicin, iodoRTX (an antagonist that acts as an agonist upon dissociation of iodine), and related potent agonists and their analogs.

Preferably, the dosage rate is optimized so as to provide an amount sufficient to selectively induce nerve terminal depolarization block and/or nerve terminal death in TRPV1-containing neurons to provide the desired pain relief without permanently damaging a significant proportion of the cell bodies, thereby allowing the nerve terminals, over time, to regenerate to obtain essentially normal nerve function in the treatment area. Accordingly, it is preferable to use the lowest concentration of agonist effective to provide the desired pain relief without causing significant permanent damage to cell bodies. Upon administration of agonist it is preferable that at least 80% of the nerve cell bodies will remain intact. More preferably, at least 90% of the nerve cell bodies will remain intact, and most preferably, essentially all of the nerve cell bodies will remain intact. Higher concentrations of agonist may be used, particularly in cases of extreme pain caused by a terminal condition (for example, bone cancer), to provide the desired pain relief with an acceptable amount of damage to nerve cell bodies.

Thus, in one form, the method of the present invention comprises contacting low concentrations of RTX to select TRPV1-containing neurons to reduce nociceptive transmission by inducing selective nerve terminal depolarization block in the short term and nerve terminal death in the long term without permanently damaging cell bodies in the TRPV1-containing neurons.

In one preferred form, concentrations of RTX, preferably in the range of about 0.01 micrograms/kilogram to about 5 micrograms/kilogram, and more preferably in the range of about 0.01 micrograms/kilogram to about 0.5 micrograms/kilogram, are introduced into a patient, preferably in a volume of about 0.01 to 0.1 milliliters of a pharmaceutically acceptable carrier, by intrathecal administration to the appropriate region of the spinal cord over a relatively short time period (e.g., about 5-15 minutes). Administration of RTX in this manner is appropriate, for example, for treating chronic inflammatory pain and neuropathic conditions, such as cancer pain (particularly bone cancer), visceral pain, and other pain involving large areas such as muscle, gut, and bone.

Alternatively, concentrations of RTX, preferably in the range of about 0.01 micrograms/kilogram to about 5 micrograms/kilogram, and more preferably in the range of about 0.01 micrograms/kilogram to about 0.5 micrograms/kilogram may be introduced by intraarticular administration, preferably in a volume of about 0.01 to 0.1 milliliters of a pharmaceutically acceptable carrier, to relieve pain involving joints, such as arthritic pain. In another form, very low concentrations of RTX, preferably about a 5-50 nM solution, may be sprayed, or otherwise applied topically, to the affected region of the patient for treatment of pain such as burn pain and pain associated with Herpes zoster and AIDS.

According to the method of the present invention, nerve terminals, especially the central terminals, can be selectively targeted by intrathecal administration of RTX, which ablates the nerve terminals and reduces TRPV1-mediated nociceptive transmission. This method allows the DRG neuronal cell body to remain intact and facilitates the regrowth of nerve terminals. Permanent damage to TRPV1-containing neurons is, therefore, minimized or avoided. Hence, the present method provides an advantage over current methods in which the neuronal cell bodies are permanently damaged and unable to regenerate, resulting in a group of neurons being permanently eliminated from the body.

The method of the present invention permits treatment of inflammatory pain conditions with a long duration of action. Thus, it is useful in treating numerous chronic pain conditions, including, for example, cancer pain, muscle pain, burn pain, visceral pain, pain of unknown origin, and the like.

Peripheral terminals may be targeted to relieve burn pain or the pain, itch, and irritation caused by various skin diseases. For example, one useful approach is to use low concentrations of RTX to cause peripheral nerve terminal ablation in burn patients who suffer from intense pain. Over time both the skin tissue as well as sensory nerve terminals will regrow.

The method of the present invention also minimizes discomfort to the patient while the TRPV1 agonist is administered. As noted above, it is thought that RTX's ability to induce slow and sustained activation of TRPV1 at lower concentrations might contribute to the lack of pungency of RTX, as compared to capsaicin. Use of iodoRTX, which acts as antagonist until iodine dissociates from the RTX, may additionally minimize discomfort during administration.

The selectivity of RTX action on TRPV1 containing nociceptive nerve terminals is likely to have a higher therapeutic index compared to other clinically available agents (toxins), such as, botulinum toxin and omega-conopeptide, which act on synaptic vesicles and Ca²⁺ channels, respectively. Thus, an effective and clinically safe protocol can be developed to treat intractable chronic pain conditions, which plague millions of people, with RTX, while minimizing permanent damage to the nerves.

The following examples describe and illustrate the processes and products of the present invention. These examples are intended to be merely illustrative of the present invention, and not limiting thereof in either scope or spirit. Those skilled in the art will readily understand that variations of the materials, conditions, and processes described in these examples can be used. All references cited herein are incorporated by reference.

### EXAMPLES

### Methods

Rat TRPV1 dcRNA. Cloned rat TRPV1 was obtained from the Julius Lab at the University of California San Francisco. TRPV1 cRNA was obtained using standard techniques known to those of skill in the art.

Electrophysiology. Whole-cell and single-channel currents were recorded from rat DRG neurons in culture and from Xenopus laevis oocytes injected with rat TRPV1 cRNA. Animals were cared for according to the standards of the National Institutes of Health (NIH). All animal use protocols were approved by Southern Illinois University School of Medicine Animal Care Committee.

Oocytes were obtained by an abdominal incision after anaesthetizing the frog by immersion in a 0.05% solution of 3-aminobenzoic acid ethyl ester (MS222). Animals were killed by a subcutaneous injection of a 2% solution of MS222 according to NIH guidelines. One day after separating the oocytes from the follicular layer, 50-70 nl TRPV1 cRNA was injected using a Drummond Nanoject (Drummond Scientific Co., Broomall, Pa., USA). Oocytes were used for recording from 3 days after the injection. Double-electrode voltage clamp was performed using a Warner amplifier (Warner Instruments, Hamden, Conn., USA). Data were digitized and stored on videotape or directly stored on the computer using a LabView interface (National Instruments, Austin, Tex., USA). Experiments were performed at 21-23° C. Oocytes were placed in a Perspex chamber superfused (5-10 ml min⁻¹) with Ca²⁺-free Ringer solution containing (mM): NaCl 100, KCl 2.5 and Hepes 5; pH adjusted to 7.35 with NaOH. Current-voltage relationships were measured using 1-s voltage ramps from −80 to +80 mV.

Primary DRG neuronal cultures were prepared from embryonic day 18 (E18) rat embryos. Adult pregnant rats were killed with an overdose of isoflurane. DRG were dissected and the cells were dissociated by triturating with a fire-polished glass pipette. Cells were cultured in Neurobasal medium (Life Technologies, Buffalo, N.Y., USA), supplemented with 10% fetal bovine serum (FBS), and grown on poly-D-lysine-coated glass coverslips. Cells were used from 5 to 15 days after plating. Small diameter (<30 μm) rounded neurons were selected for patch-clamp experiments. More than 70% of the neurons responded to capsaicin. The Giga-seal patch-clamp technique was used to record whole-cell currents. For perforated-patch recordings, the bath solution contained (mM): sodium gluconate 140, KCl 2.5, Hepes 10, MgCl₂ 1 and ethylene glycol bis(2-aminoethyl ether)-N,N,N′N′-tetraacetic acid (EGTA) 1.5 (pH adjusted to 7.35 with NaOH), and the pipette solution contained (mM): sodium gluconate 130, NaCl 10, KCl 2.5, Hepes 10, MgCl₂ 1, EGTA 1.5 and 240 μg/ml⁻¹ amphotericin B (pH adjusted to 7.35 with NaOH). For current-clamp experiments the pipette solution contained (mM): potassium gluconate 130, NaCl 10, MgCl₂ 1, EGTA 0.2, K₂ATP 1 and Hepes 10; pH adjusted to 7.35 with NaOH. Extracellular solution for current- and voltage-clamp experiments contained (mM): NaCl 140, KCl 4, MgCl₂ 1 and Hepes 10; pH adjusted to 7.35 with NaOH. Currents were recorded using a WPC 100 patch-clamp amplifier (E.S.F. Electronic, Goettingan, Germany). Data were filtered at 10 kHz, digitized (VR-10B, Instrutech Corp., Great Neck, N.Y., USA) and stored on videotapes or directly stored in the computer using a LabView interface. For analysis of whole-cell currents, data were filtered at 1 kHz (−3 db frequency with an 8-pole low-pass Bessel filter, Warner Instruments, LPF-8) and digitized at 2 kHz.

For single-channel recording in cell-attached patches from DRG neurons, the bath solution contained (mM): potassium gluconate 140, KCl 2.5, MgCl₂ 1, Hepes 5 and EGTA 1.5; pH adjusted to 7.35 with NaOH. The patch pipettes were made from glass capillaries (Drummond, Microcaps), coated with Sylgard (Dow Corning, Midland, Mich., USA). For cell-attached patches, the patch pipettes were filled with a solution that contained (mM): sodium gluconate 140, NaCl 10, MgCl₂ 1, Hepes 5 and EGTA 1.5; pH adjusted to 7.35 with NaOH. While recording from cell-attached patches, in order to avoid differences in the driving force because of the differences in the membrane potential, the external NaCl was replaced by an equal amount of KCl in order to nullify the membrane potential. For outside-out patches, the pipette solution contained (mM): sodium gluconate 90, NaCl 10, BAPTA 10, Hepes 10, K₂ATP2 and GTP 0.25; pH adjusted to 7.35 with NaOH. The bath solution contained (mM): sodium gluconate 100, KCl 2.5, MgCl₂ 1, Hepes 5 and EGTA 1.5; pH adjusted to 7.35 with NaOH. All the experiments were performed at room temperature (21-23° C.). Agar-bridge electrodes were used to avoid changes in junction potential. The currents were recorded using a WPC 100 (Warner Instruments) or Axopatch 2B (Axon Instruments, Union City, Calif., USA) patch-clamp amplifier. Data were filtered at 10 kHz (Axopatch 2B), digitized (VR-10B, Instrutech Corp.), and stored on videotapes. For the analysis of amplitude and open probability (Pₒ), the data were filtered at 2.5 kHz (−3 db frequency with an 8-pole low-pass Bessel filter, Warner Instruments) and digitized at 5 kHz. For dwell-time analysis, the data were filtered at 10 kHz and digitized at 50 kHz.

Single-channel analysis. Single-channel current amplitude and Pₒ were estimated from all-point current amplitude histograms (Channel 2 software kindly provided by Michael Smith, Australian National University, Can berra, Australia) and fitted to Gaussian densities (Origin, OriginLab Corp., Northampton, Mass., USA). For current-voltage relationships, the amplitudes were determined by fitting a Gaussian curve to an all-point histogram. Pₒ was determined using unedited segments of data, which were typically 1-5 min long. For multiple channel patches, the mean Pₒ was measured as nPₒ, where n is the number of channels in the patch. Chord conductance was measured at +60 or −60 mV. Slope conductance was determined by linear fits to current-voltage data between +20 and +100 mV or −20 and −100 mV.

Patches that apparently had a single TRPV1 channel (assessed by the lack of overlapping events at +60 mV, when the Pₒ was >0.7) were used for dwell-time analysis. Single-channel currents were idealized using a modified Viterbi algorithm (QUB software, www.qub.buffalo.edu). Dwell-time distributions were fitted with mixtures of exponential densities using a method of maximum likelihood. Additional exponential components were incorporated only if the maximum log likelihood increased more than 2 log likelihood units. (Chung et al. 1990; Qin et al. 1996; Premkumar et al. 1997.) A dead time (T_{d}) of 50 μs was imposed retrospectively, in that, events shorter than 50 μs were considered to be a part of the adjoining sojourns.

All the chemicals used in these experiments were obtained from Sigma (St. Louis, Mo., USA). The working concentrations of the drugs were freshly prepared from the following ethanol stock solutions: RTX (100 mM), capsaicin (100 mM) and capsazepine (50 mM). The final solution contained <0.001% ethanol. Data are given as mean±S.E.M and statistical significance was evaluated using the Student's t test.

### Example 1

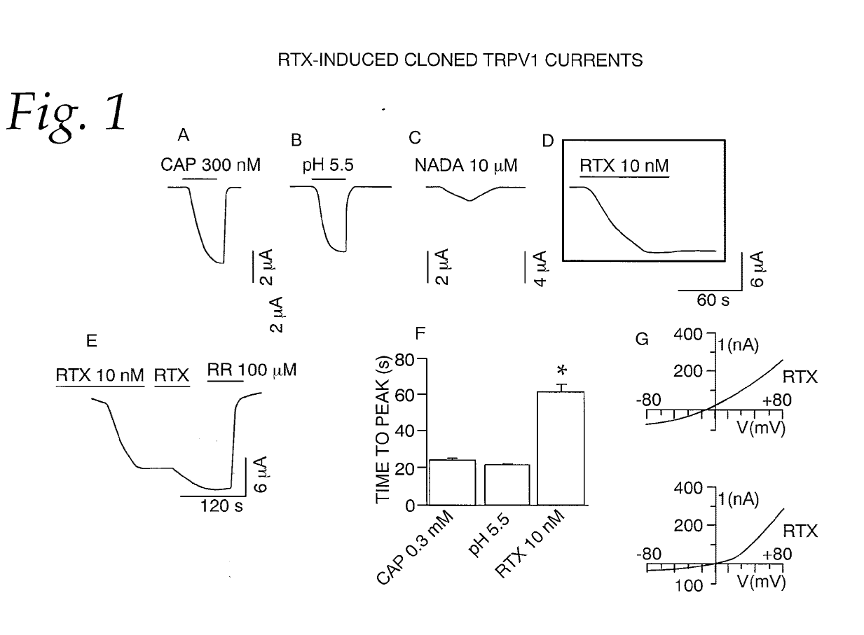

This example tests the activation of whole-cell currents in oocytes by RTX. Whole-cell currents were recorded from oocytes heterologously expressing TRPV1. At a holding potential of −60 mV, application of capsaicin (300 nM), protons (pH 5.5), N-arachidonyl dopamine (NADA) (10 μM) and RTX (10 nM) induced inward currents. NADA is a weak agonist and did not induce a maximal response even at a concentration of 10 μM. The currents induced by capsaicin, protons and NADA could be reversed readily when the agonists were removed (FIG. 1A-C). In contrast, RTX-induced currents did not deactivate even after a prolonged washout (>15 min) (FIG. 1D). Qualitatively, it is clear that the activation and deactivation phases are different for RTX-induced currents as compared to currents activated by capsaicin, protons and NADA. (FIG. 1A, B and C). RTX-induced currents are activated slowly and minimally deactivated (FIGS. 1D and E). Moreover, repeated application of a submaximal concentration of RTX (10 nM) induced larger currents until a maximal response was attained; the current could be readily blocked by ruthenium red (100 μM) (FIG. 1E). To quantify the differences in activation phase, we measured the activation time (10-90% of the rising phase) and the summary graph shows that the activation phase of RTX-induced currents is two-fold slower than capsaicin-induced (300 nM) and proton-induced (pH 5.5) currents (capsaicin, 23.9±1.5 s, n=11; protons, 20.2±0.7 s, n=11; RTX, 61.4±4.4 s, n=13) (FIG. 1F). A ramp protocol, which changed the voltage from −80 to +80 mV in 1s, was used to determine the current-voltage relationship of the responses induced by RTX (10 nM) and capsaicin (300 nM) (FIG. 1G). Agonist-induced currents are shown after subtracting the leak current in the absence of the agonist. Capsaicin- and RTX-induced currents reversed at −12.3±0.42 (n=3) and −10.4±0.53 (n=4) mV, respectively. Although an outward rectification pattern was seen with both agonists, RTX-induced currents showed a lesser degree of outward rectification. In order to quantify this, we calculated the ratio of outward and inward currents. The ratio was significantly larger (P<0.05) for capsaicin-induced (6.6±1.3, n=6) as compared to RTX-induced (2.35±0.97, n=3) currents. Although increasing concentrations of RTX induced larger currents, repeated application of the same sub-maximal concentration also induced larger currents. Thus, it was difficult to construct a meaningful dose-response curve for RTX. Repeated application of agonists such as capsaicin, protons, NADA and anandamide increases the amplitude of the current in oocytes and DRG neurons; in the case of NADA and anandamide this has been attributed to activation of PKC. PKC activation increases sensitivity as well as TRPV1 translocation. (Van Buren et al. 2005.) A similar phenomenon could play a role in the observation of increased current amplitude with repeated application of low concentrations of RTX. (Premkumar et al. 2004.) It is also possible that RTX is partitioned in the membrane and repeated application can increase the availability of RTX for binding. In the past, an EC₅₀ value (39 nM) has been obtained by applying increasing concentrations of RTX; however, results from this study suggest that a dose-response curve does not give an accurate determination of the EC₅₀ value, but it does confirm the ultra potency of RTX. (Szallasi et al. 1990b; Caterina et al. 1997; Marshall et al. 2003.)

### Example 2

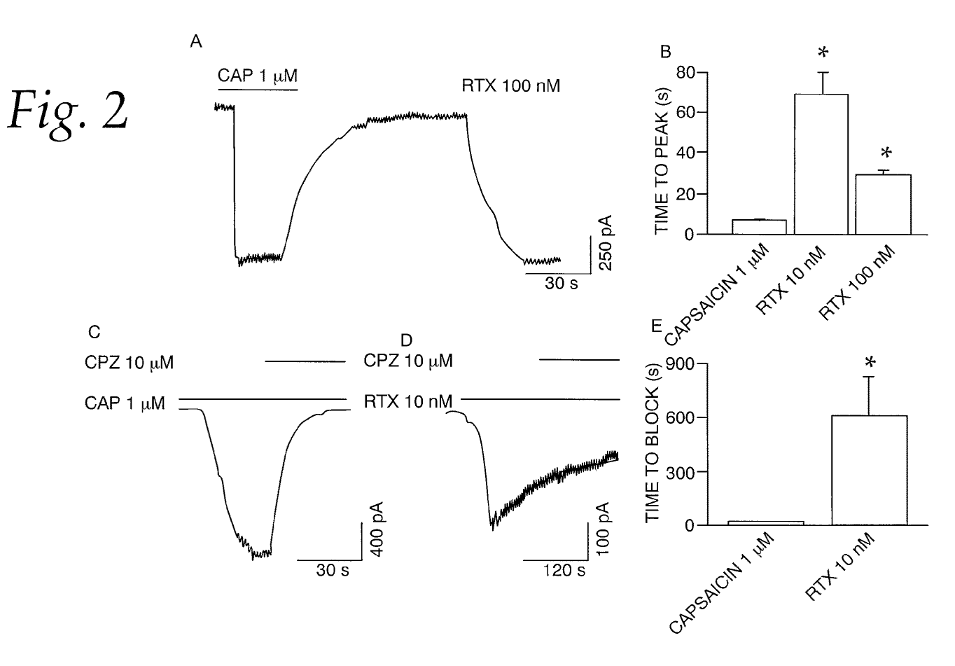

This example tests the activation of whole-cell current in DRG neurons by RTX. We determined the properties of RTX-induced membrane currents on native TRPV1 in embryonic DRG neurons grown in culture. The cells were voltage-clamped at −60 mV and currents were evoked by RTX and capsaicin. Protons were not used to elicit currents because of the presence of acid-sensitive ion channels in these neurons. Capsaicin-induced (1 μM) currents were readily reversible. However, as previously observed in oocytes (FIG. 1D), RTX (10 and 100 nM) induced a sustained current that could not be reversed readily even after a prolonged washout (>15 min) (FIG. 2A). Furthermore, capsaicin-induced currents exhibited a relatively fast activation and deactivation phase, whereas RTX-induced currents exhibited significantly slower activation phase as compared to capsaicin-induced currents (RTX 10 nM, 69±10.9, n=6; RTX 100 nM, 29.35±2.65 s, n=6; capsaicin 1 μM, 7.14±0.42 s, n=66) (P<0.001, FIGS. 2A and B). We did not observe any difference in the activation kinetics of RTX-induced currents with or without prior application of capsaicin.

We determined the block of TRPV1 current induced by RTX and capsaicin by a competitive TRPV1 blocker. Capsazepine (10 μM) completely blocked the current induced by capsaicin (1 μM) and RTX (10 nM). However, the rate of block induced by capsazepine was significantly slower (612.3±219 s, n=6) for RTX-induced currents than for capsaicin-induced currents (18.4±2.8 s, n=10) (FIG. 2C-E, P<0.01). These experiments suggest that even though RTX binds to TRPV1 with high affinity, it is unable to activate the receptor rapidly and deactivates minimally.

### Example 3

This example tests the single channel currents activated by RTX and capsaicin in native and cloned TRPV1.

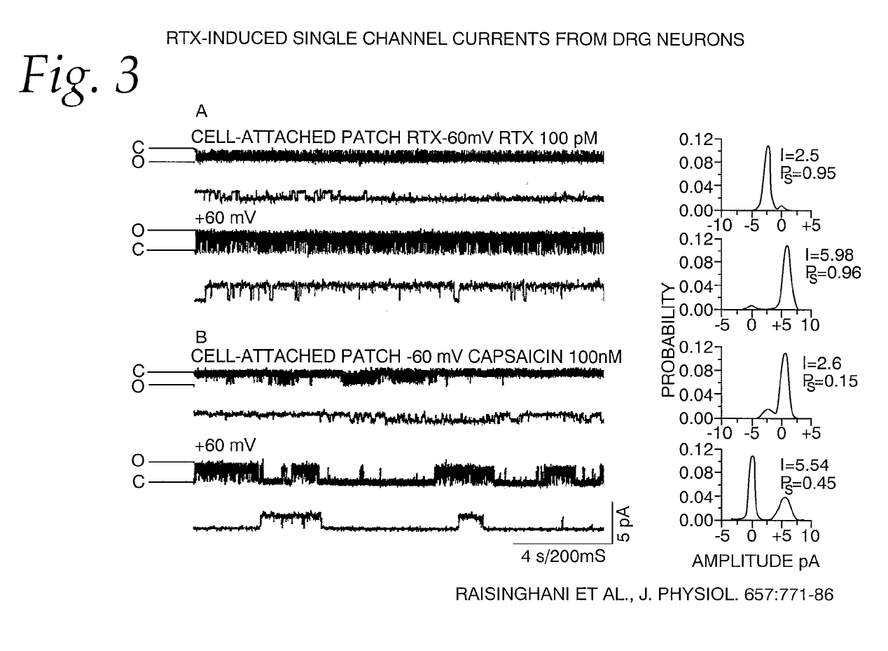

Single-channel conductance. To further evaluate the properties of TRPV1 activation by RTX, we recorded single-channel currents from cell-attached and excised patches from DRG neurons and oocytes heterologously expressing TRPV1. All recordings were carried out in the absence of extracellular calcium to avoid tachyphylaxis and desensitization of channel activity. (Docherty et al. 1996; Caterina et al. 1997; Koplas et al. 1997.) Single-channel current activity in cell-attached patches from DRG neurons were recorded at −60 and +60 mV, as shown in FIG. 3. Single-channel current amplitude of RTX-induced currents was 2.3±0.21 pA (n=8) at −60 mV and 6.02±0.14 pA (n=6) at +60 mV corresponding to a conductance of 38.3±3.5 pS and 100±2.2 pS, respectively (FIG. 3A). Although RTX is a potent agonist, single-channel conductance is lower at negative potentials as compared to positive potentials. In comparison to RTX-induced currents, single-channel currents induced by capsaicin (100 nM) had amplitudes of 2.4±0.12 (n=10) and 5.67±0.06 pA (n=10) at −60 and +60 mV corresponding to conductances of 40±1.95 and 95±0.1 pS, respectively (FIG. 3B, also see Premkumar et al. 2002).

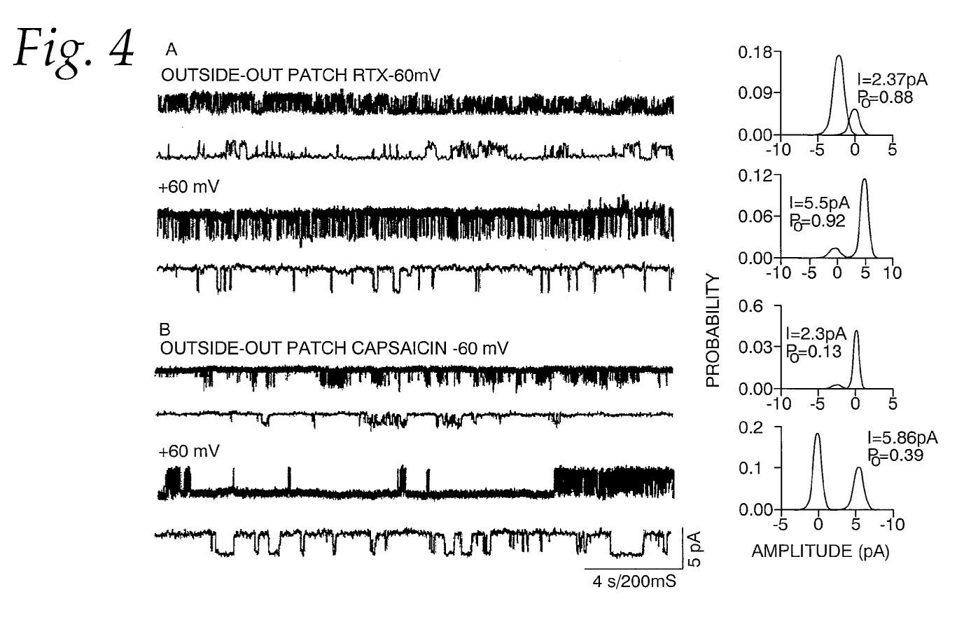

Next we recorded single-channel currents from the cloned TRPV1 in excised patches. RTX-induced current amplitudes were 2.59±0.17 and 6.19±0.05 pA (n=6) corresponding to conductances of 43±2.8 and 103.3±0.83 pS, at −60 and +60 mV, respectively (FIG. 4A). Capsaicin-induced current amplitudes were 2.52±0.08 (n=27) and 5.71±0.1 pA (n=28) corresponding to conductance of 41.8±1.3 and 95.3±1.7 pS at −60 mV and +60 mV, respectively, as previously described (FIG. 4B). (Premkumar et al. 2002.)

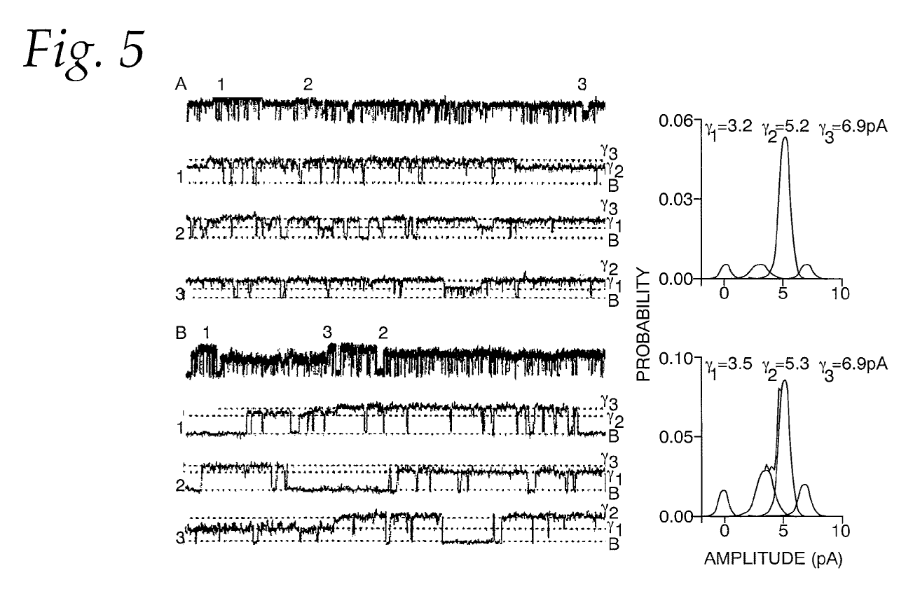

Careful analysis of single-channel currents induced by RTX revealed multiple conductance states. Both, supra- and subconductance states were observed (FIGS. 5A and B). In comparison to capsaicin-induced currents, which predominantly dwelled at a conductance level of ˜95 pS, RTX-induced currents showed multiple conductance states. In DRG neurons at +60 mV, the current amplitudes were 3.2, 5.2 and 6.9 pA, corresponding to conductance levels of 53, 86 and 115 pS, respectively (FIG. 5A). In oocytes expressing TRPV1, at +60 mV, RTX-induced currents had amplitudes of 3.5, 5.3 and 6.9 pA, corresponding to single-channel conductance of 58, 88 and 115 pS, respectively (FIG. 5B).

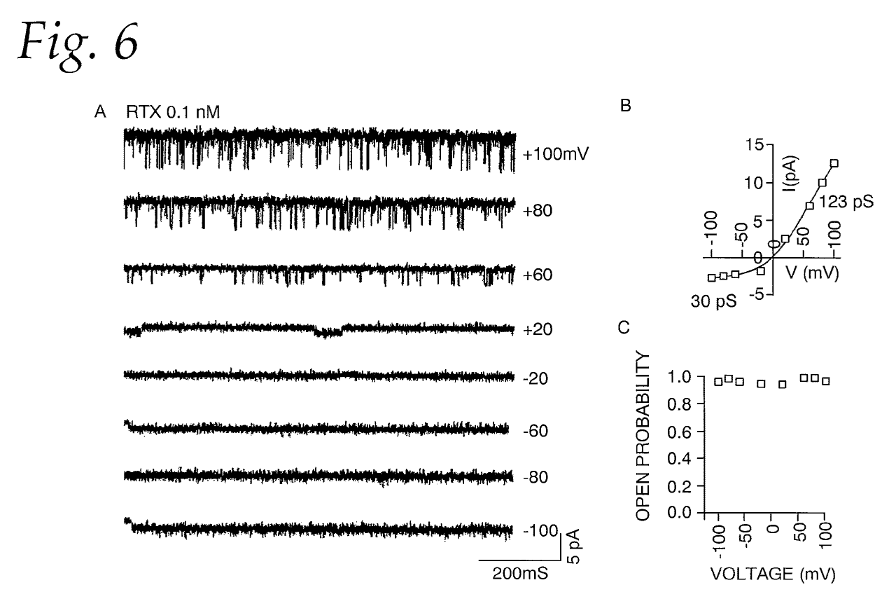

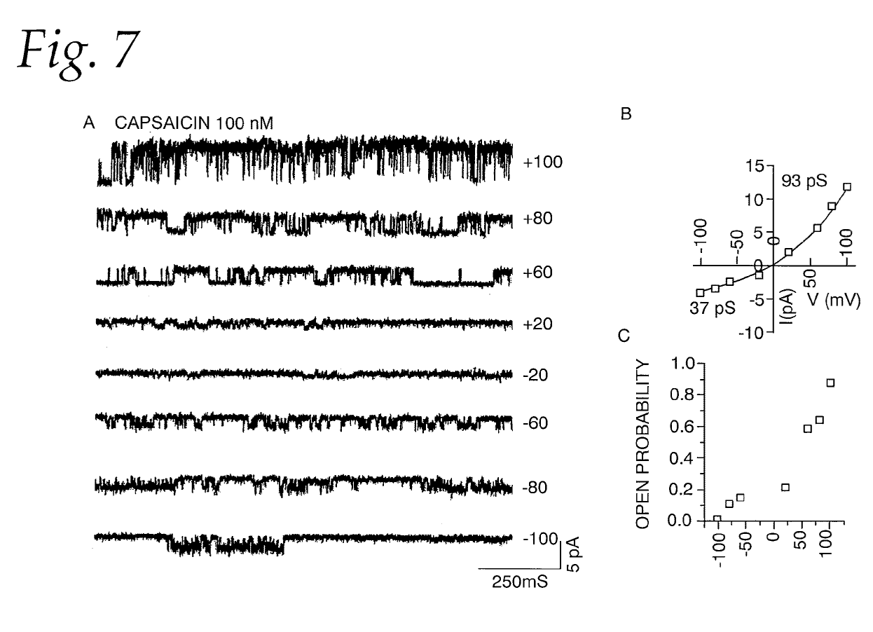

Current-voltage relationship and rectification. RTX- and capsaicin-induced channel activity were recorded from −100 to +100 mV (FIG. 6). Several studies have shown that the whole-cell current-voltage relationship for TRPV1 exhibits a profound outward rectification. (Caterina et al. 1997; Tominaga et al. 1998; Premkumar et al. 2002.) For RTX-induced currents, the polarity of single-channel currents reversed close to 0 mV (−2.9 mV, n=2). The outward limb of the single-channel current-voltage relationship has a slope conductance of 126 pS (between 0 and +100 mV) and the inward current limb has a slope conductance of ˜30 pS (between 0 and −100 mV) (FIGS. 6A and B). At negative potentials, single-channel conductance did not change linearly with voltage beyond −60 mV, whereas at positive potentials the current-voltage curve was ohmic. We do not have a good explanation for this observation. Similar to previous studies, this study showed that the slope conductance of capsaicin-induced currents is 41 pS at negative potentials as compared to 107 pS at positive potentials (FIGS. 7A and B). Single-channel conductance is lower at negative than at positive potentials and increased linearly with voltage at both negative and positive potentials. Even though RTX is a potent activator, the current-voltage relationship is not linear, suggesting that the difference in conductance is an inherent property of the receptor. The difference in single-channel conductance is partly responsible for the outward rectification, but to a lesser extent as compared to capsaicin-induced currents, which also exhibits is due to a lack of voltage-dependent change in Pₒ as described below.

Single-channel open probability (Pₒ). Single-channel activity of RTX-induced currents in cell-attached patches as well as outside-out patches exhibited a high Pₒ (˜0.9) (FIGS. 3A, 4A and 6A). Single-channel Pₒ did not change with increasing concentrations of RTX from 0.1 to 20 nM. Only the time to attain maximum Pₒ was longer when lower concentrations of RTX were used. Thus, all single-channel analyses for RTX-induced activation of TRPV1 were performed in the presence of 0.1-0.2 nM RTX. Moreover, there is no voltage-dependent change in Pₒ, unlike capsaicin-induced currents. At both negative and positive potentials, the Pₒ was 0.9 (FIGS. 3A, 4A, and 6A and C). Single channel Pₒ was 0.94±0.02 (n=6) and 0.98±0.02 (n=3) at −60 and +60 mV, respectively, in cell-attached patches from DRG neurons (FIG. 3A).

Next we recorded single-channel currents using outside-out patches from oocytes expressing TRPV1. In the presence of RTX, the Pₒ was high: 0.83±0.05 (n=3) and 0.88±0.03 (n=3) at −60 and +60 mV, respectively (FIG. 4A). Even in excised patches, RTX-induced Pₒ is higher than capsaicin-induced Pₒ.

Application of capsaicin showed a concentration-dependent increase and a voltage-dependent decrease in Pₒ at negative potentials. (Premkumar et al. 2002.) In cell-attached patches, the Pₒ in the presence of 100 nM capsaicin at −60 and +60 mV was 0.1±0.04 (n=6) and 0.53±0.04 (n=7), respectively (FIGS. 3B, 4B and 7A and C). Similarly, in excised patches the Pₒ in the presence of 100 nM capsaicin at −60 and +60 mV was 0.14±0.01 (n=34) and 0.4±0.03 (n=38), respectively. The maximal Pₒ in the presence of 1 μM capsaicin was 0.8±0.04 and 0.4±0.08 in cell-attached patches at −60 and +60 mV, respectively, as reported in a previous study. (Premkumar et al. 2002.) Even at high concentrations of capsaicin, the voltage-dependent change in Pₒ at negative potentials was apparent. Moreover, capsaicin-induced TRPV1 channel activity was reversible even at higher concentrations, unlike the irreversible nature of activation observed with RTX (data not shown), which was consistent with the observation in whole-cell experiments.

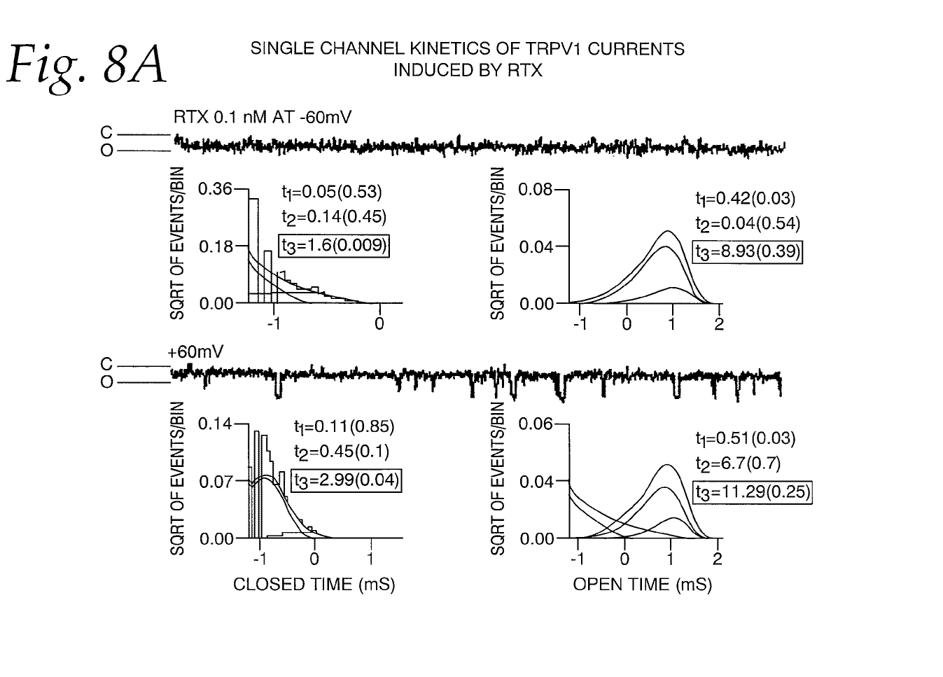

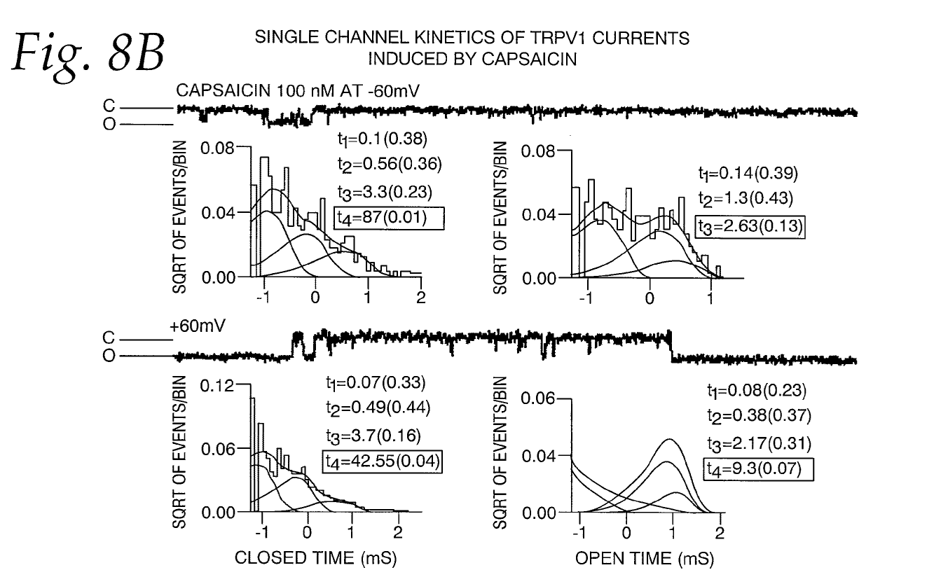

Single-channel kinetics of RTX- and capsaicin-induced channel activity. Single-channel kinetic analyses were carried out from patches that apparently had one channel, using the criterion of non-overlapping events when the Pₒ was >0.7. The data (stretches >1 min) were first idealized using a single open and closed state. This was followed by incorporation of additional open and closed states until the dwell-time histograms were well-fitted with a mixture of exponential densities. Method of maximal log likelihood was used to determine the best fit for the data. Additional states were incorporated only if the log likelihood increased by at least 2 log units. Analyses of single-channel kinetics of TRPV1 currents activated by RTX exhibited no voltage-dependent behavior, unlike capsaicin-induced currents (Table 1, FIG. 8).

The open-time distribution of RTX-induced (0.1-0.2 nM) single-channel currents recorded in DRG neurons could be well-fitted with three exponential components (FIG. 8A and Table 1).

Increasing the concentration of RTX did not change the time constants or their relative areas of distribution, unlike with capsaicin. (Premkumar et al. 2002.) This finding is consistent with the observation that Pₒ is independent of the concentration. The mean open-time constants and their relative areas of distribution (in parentheses) in the presence of RTX at −60 mV in cell-attached patches were 0.46±0.1 ms (0.03±0.01), 5.57±2.4 ms (0.5±0.3) and 10.2±1.8 ms (0.47±0.3) (n=6). The mean open-time constants and their relative areas of distribution at +60 mV were 1.6±1.2 ms (0.11±0.1), 6.58±0.56 ms (0.72±0.12) and 12±1 ms (0.25±0.01) (n=3) (FIG. 8A right panel and Table 1). The mean open times of RTX-induced currents are 7.5 ms and 7.9 ms at −60 and +60 mV, respectively. From these analyses, it is clear that RTX-induced currents dwelled predominantly in long open states.

TABLE 1
Open and closed time distribution of RTX- and capsaicin-induced currents

| Vₘ | | | Conc. | Open time | | | |
|---|---|---|---|---|---|---|---|
| (mV) | n | Pₒ | (nM) | τ1 | τ2 | τ3 | τ4 |

RTX

| | | | | | | | |
|---|---|---|---|---|---|---|---|
| −60 | 6 | 0.94 ± 0.02 | 0.1 | 0.46 ± 0.1 | 5.57 ± 2.4 | 10.2 ± 1.8 | — |
| | | | | (0.03 ± 0.01) | (0.5 ± 0.3) | (0.47 ± 0.3) | — |
| +60 | 3 | 0.98 ± 0.02 | 0.1 | 1.6 ± 1.2 | 6.58 ± 0.56 | 12 ± 1 | — |
| | | | | (0.11 ± 0.1) | (0.72 ± 0.12) | (0.25 ± 0.01) | — |

CAP

| | | | | | | | |
|---|---|---|---|---|---|---|---|
| −60 | 6 | 0.1 ± 0.04 | 100 | 0.11 ± 0.01 | 0.76 ± 0.18 | 2.61 ± 0.84 | — |
| | | | | (0.26 ± 0.05) | (0.49 ± 0.07) | (0.34 ± 0.08) | — |
| +60 | 7 | 0.53 ± 0.04 | 100 | 0.14 ± 0.04 | 0.7 ± 0.13 | 3.83 ± 0.62 | 14.7 ± 2.6 |
| | | | | (0.11 ± 0.02) | (0.28 ± 0.03) | (0.44 ± 0.02) | (0.13 ± 0.03) |

| Vₘ | Closed time | | | |
|---|---|---|---|---|
| (mV) | τ1 | τ2 | τ3 | τ4 |
| RTX | | | | |
| −60 | 0.05 ± 0.007 | 0.12 ± 0.005 | 1.1 ± 0.26 | — |
| | (0.47 ± 0.02) | (0.5 ± 0.02) | (0.01 ± 0.002) | — |
| +60 | 0.1 ± 0.008 | 0.46 ± 0.02 | 8.7 ± 3.9 | — |
| | (0.89 ± 0.02) | (0.08 ± 0.01) | (0.01 ± 0.01) | — |
| CAP | | | | |
| −60 | 0.13 ± 0.02 | 0.79 ± 0.12 | 3.47 ± 0.59 | 30.8 ± 12.4 |
| | (0.43 ± 0.04) | (0.31 ± 0.03) | (0.19 ± 0.03) | (0.04 ± 0.02) |
| +60 | 0.06 ± 0.02 | 0.67 ± 0.39 | 1.66 ± 0.39 | 70.84 ± 47 |
| | (0.39 ± 0.04) | (0.4 ± 0.03) | (0.16 ± 0.01) | (0.04 ± 0.009) |

*The numbers in parentheses represent the relative areas of distribution.

For capsaicin-induced currents, at negative potentials (−60 mV), three exponential components were required to fit the open-time distributions. The mean open-time constants and their relative areas of distribution at −60 mV were 0.11±0.01 (0.26±0.05), 0.76±0.18 (0.49±0.07), 2.61±0.84 (0.34±0.08) (n=6). At positive potentials four exponential components were required. The mean open-time constants and their relative area of distribution at +60 mV were 0.14±0.04 (0.11±0.02), 0.7±0.13 (0.28±0.03), 3.83±0.62 (0.44±0.02) and 14.7±2.6 (0.13±0.03) (n=7) (FIG. 8B right panel and Table 1). The mean open times of capsaicin-induced currents are 1.27 and 3.8 ms at −60 and +60 mV, respectively. It is clear from this analysis that in the presence of RTX, the open-time distributions and the mean open time did not show any voltage dependence, unlike in the presence of capsaicin. Also, in the presence of capsaicin the mean open times are shorter at −60 and +60 mV.

In most of the patches, the closed-time distribution of RTX-induced currents could be well-fitted with three exponential components. The fractional area of the third exponential component was negligible (FIG. 8A left panel and Table 1). The closed-time constants and their fractional area of distribution at −60 mV in cell-attached patches were 0.05±0.007 (0.47±0.02), 0.12±0.005 (0.5±0.02), 1.1±0.26 (0.01±0.002) (n=6). The closed-time constants and their relative area of distribution at +60 mV in cell-attached patches were 0.1±0.008 (0.89±0.02), 0.46±0.02 (0.08±0.01) and 8.7±3.9 (0.01±0.01) (n=3) (FIG. 8A left panel and Table 1).

In the presence of capsaicin the closed-time distribution was well fitted with four exponential components (FIG. 8B left panel and Table 1). The time constants of the two shortest exponential components changed minimally, if at all, with capsaicin concentration, suggesting that these components reflect fully liganded states. At −60 mV, the mean closed-time constants and their relative area of distribution were 0.13±0.02 (0.43±0.04), 0.79±0.12 (0.31±0.03), 3.47±0.59 (0.19±0.03) and 30.8±12.4 (0.04±0.02). At +60 mV, the mean closed-time constants and their relative areas of distribution were 0.06±0.02 (0.39±0.04), 0.67±0.39 (0.4±0.03), 1.66±0.39 (0.16±0.01) and 70.84±47 (0.04±0.009) (FIG. 8B left panel and Table 1). Analyses of closed-time distributions reveal that in the presence of RTX, almost all of the events could be fitted with only two exponential components. The two shortest closed time constants were similar to the values obtained in the presence of capsaicin irrespective of the concentration and patch configuration, suggesting that these time constants represent fully liganded closed states. (Premkumar et al. 2002.) The fractional areas of distribution of the longer closed-time constants in the presence of RTX were negligible and significantly different from those in the presence of capsaicin (P<0.05).

### Example 4

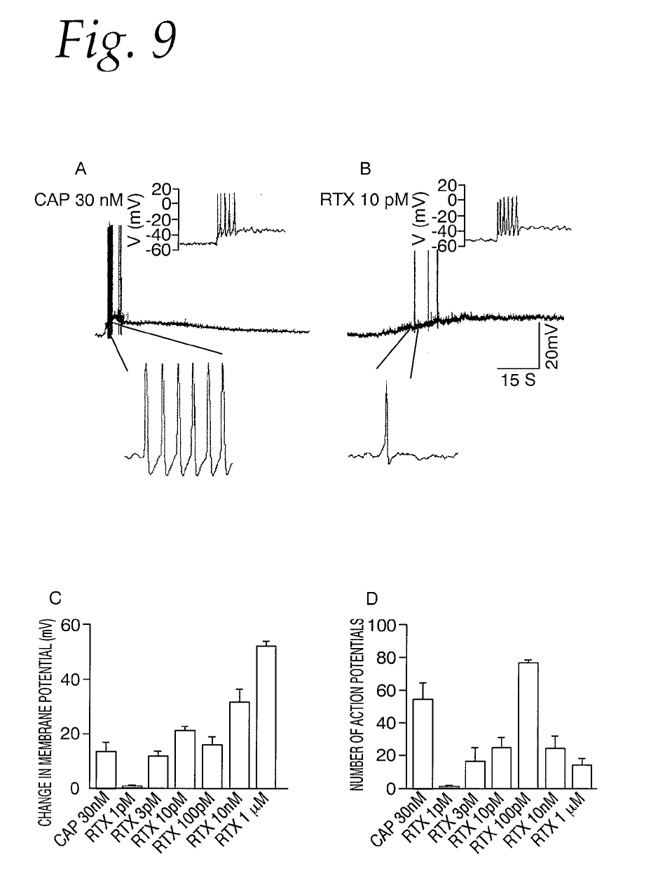

This example tests the ability of RTX and capsaicin to cause membrane depolarization and generate action potentials. To attribute a physiological significance to the slow and irreversible action of RTX, we recorded membrane depolarization in current-clamp conditions. Action potentials in response to a current injection (10-100 pA) were recorded in order to confirm that the neurons were excitable (inset to FIGS. 9A and B). The average membrane potential was −57.1±1 mV (n=42).

Capsaicin induced a dose-dependent depolarization and when it reached threshold it generated bursts of action potentials. At lower concentrations of capsaicin (<30 nM), the depolarization did not reach the threshold to fire action potentials (data not shown). Capsaicin (30 nM) induced a depolarization of 13.3±3.5 mV, n=7 (FIGS. 9A and C). The extent of membrane depolarization induced by lower concentrations of RTX was similar to that seen with capsaicin (3 pM, 10.6±1.6 mV, n=6; 10 μM, 14.1±1.5 mV, n=10; 100 μM, 16.3±3 mV, n=8). Higher concentrations of RTX induced greater depolarization (10 nM, 32.2±5.1 mV, n=5; 1 μM, 52±2 mV, n=3) (FIG. 9C).

Capsaicin (30 nM) generated a significantly greater (P<0.05) number of action potentials (54.2±10.5) as compared to lower concentrations of RTX (3 pM, 16.6±8.3, n=6; 10 pM, 24.6±6.9, n=10). At intermediate concentrations (˜100 pM) there was an increase in the number of action potentials (77.8±11.8, n=8).

At higher concentrations (10 nM-1 μM) of RTX there was a decrease in the number of action potentials probably due to rapid and sustained depolarization (10 nM, 24.5±8.6, n=5; 1 μM, 14.6±4.6, n=3) (FIG. 9D). Even though RTX induced a concentration-dependent change in the membrane potential, its effect on the number of action potentials followed a bell-shaped curve with highest activity being induced by intermediate concentrations (˜100 μM). Thus, lower concentrations of RTX can be potentially used to induce depolarization, which might be sufficient to induce a sustained Ca²⁺ influx (due to its irreversible nature) that could lead to neuronal degeneration over time and contribute to its clinical usefulness. These results suggest that because of its ultrapotent nature, low concentrations of RTX can cause a sustained activation of TRPV1 without generating action potentials, preventing the nociceptive information from reaching the brain. A similar phenomenon occurring at the nerve terminals innervating the bladder could explain why intravesicular administration of RTX causes less discomfort/pain during the treatment for bladder hyperreflexia.

### Example 5

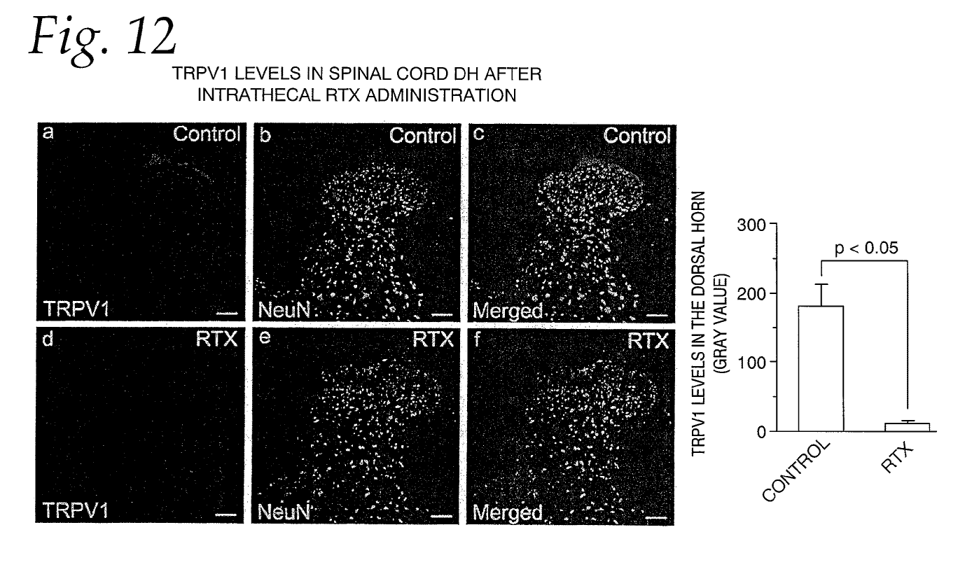

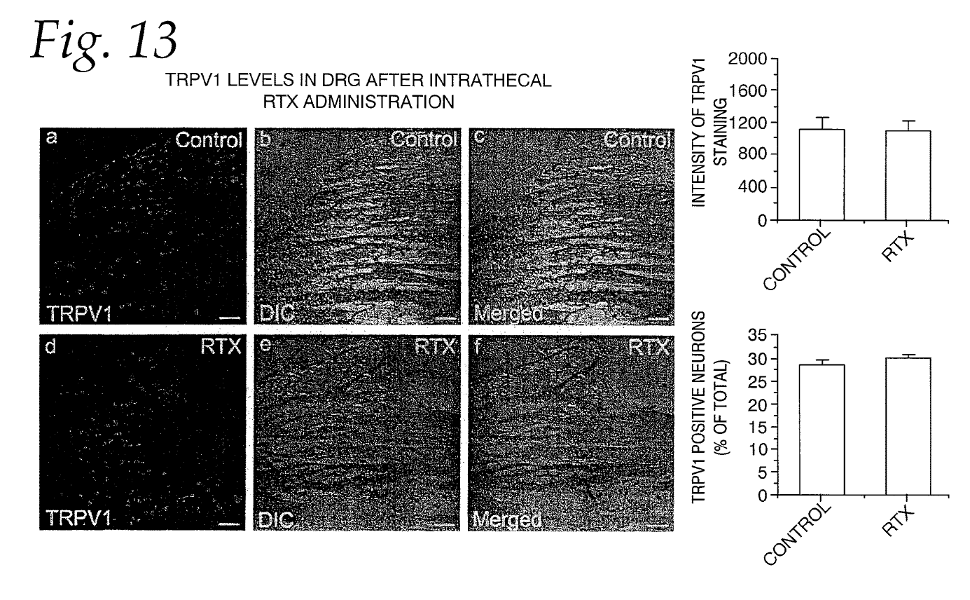

This example tests the hypothesis that ablation of TRPV1-containing central terminals can selectively alleviate inflammatory thermal pain. Low concentrations of RTX (0.19 micrograms/kg) were introduced intrathecally. After intrathecal RTX administration, TRPV1 levels in spinal cord DH were significantly reduced, indicating nerve terminal ablation (FIG. 12). Conversely, TRPV1 levels in DRG remained constant, indicating that the DRG nerve cell bodies remained intact (FIG. 13).

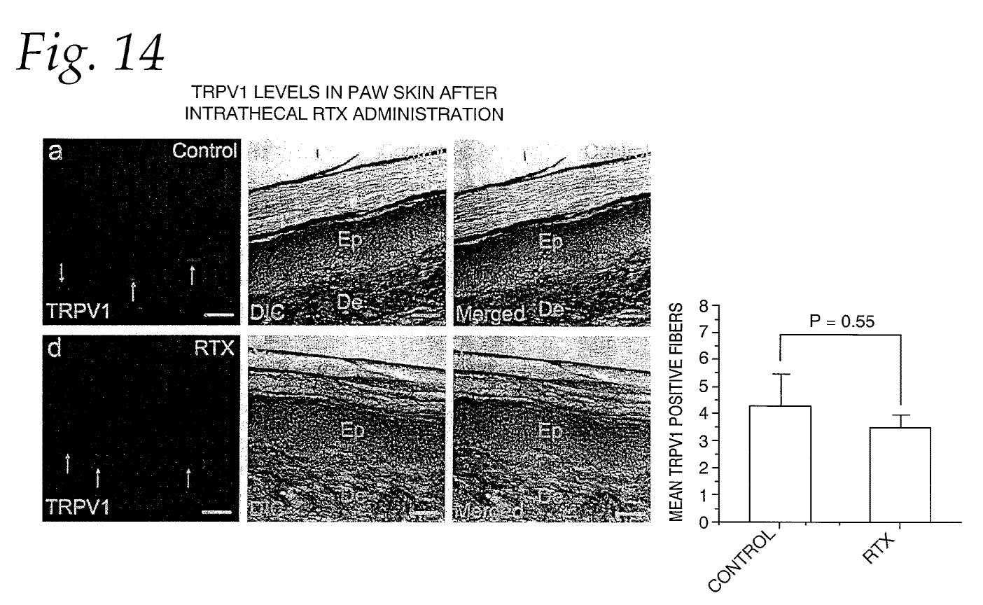

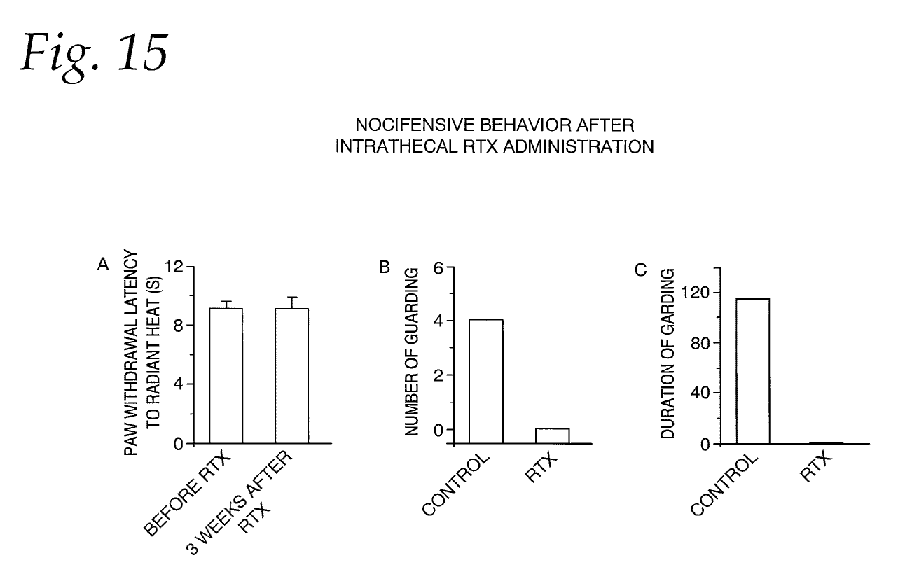

Paw withdrawal latency (PWL) to radiant heat was determined and surprisingly no change in PWL was found (FIG. 15). FIG. 14 provides photographs of TRPV1 levels in paw skin with and without intrathecal RTX administration. Thus, intrathecal administration of RTX only selectively affects central terminals without affecting the cell body or peripheral terminals. However, because PWL is a test for acute pain sensation, it is inappropriate for studying TRPV1 involvement, because TRPV1 mediates inflammatory pain.

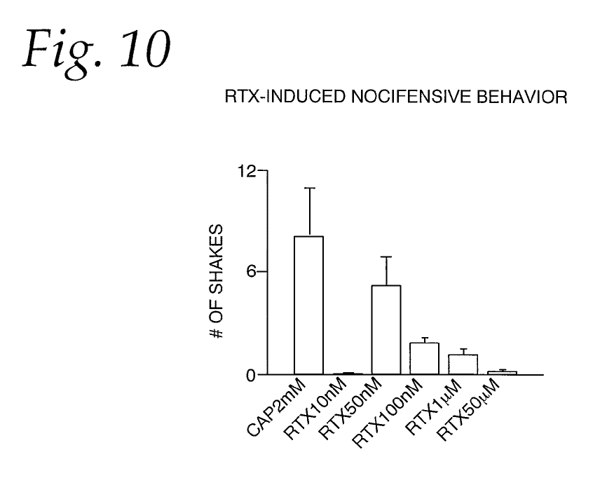

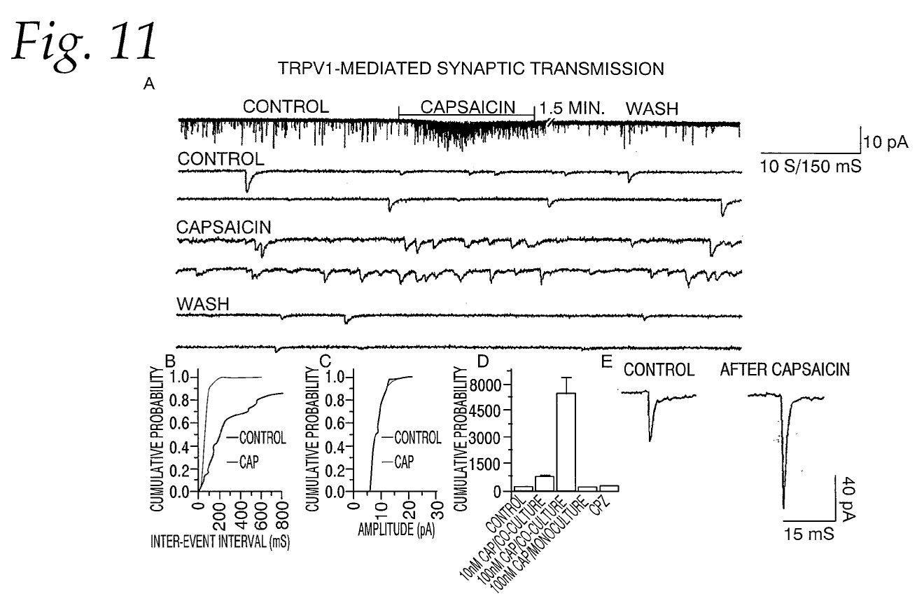

We subsequently observed nocifensive behavior to intraplantar injection of capsaicin. FIG. 10 shows RTX-induced nocifensive behavior. FIG. 11 shows TRPV1-mediated synaptic transmission. We found a dramatic decrease in nocifensive behavior as indicated by the guarding behavior and duration of guarding (FIG. 15). The number of guarding and the duration of guarding decreased significantly. This is a significant finding of this study, suggesting that ablation of TRPV1-containing central terminals can selectively alleviate inflammatory thermal pain.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

### REFERENCES

1. Acs et al. (1995). Resiniferatoxin-amide and analogues as ligands for protein kinase C and vanilloid receptors and determination of their biological activities as vanilloids. J Neurochem 65, 301-318.
2. Almasi et al. (2003). Effect of resiniferatoxin on the noxious heat threshold temperature in the rat: a novel heat allodynia model sensitive to analgesics. Br J Pharmacol 139, 49-58.
3. Baccei et al. (2003). Development of nociceptive synaptic inputs to the neonatal rat dorsal horn: glutamate release by capsaicin and menthol. J Physiol 549, 231-242.
4. Birder et al. (2002). Altered urinary bladder function in mice lacking the vanilloid receptor TRPV1. Nat Neurosci 5, 856-860.
5. Biro et al. (1997). Recent advances in understanding of vanilloid receptors: a therapeutic target for treatment of pain and inflammation in skin. J Investig Dermatol Symp Proc 2, 56-60.
6. Brady et al. (2004). Parallel changes in bladder suburothelial vanilloid receptor TRPV1 and pan-neuronal marker PGP9.5 immunoreactivity in patients with neurogenic detrusor overactivity after intravesical resiniferatoxin treatment. BJU Int 93, 770-776.
7. Caterina et al. (2001). The vanilloid receptor: a molecular gateway to the pain pathway. Annu Rev Neurosci 24, 487-517.
8. Caterina et al. (2000). Impaired nociception and pain sensation in mice lacking the capsaicin receptor. Science 288, 306-313.
9. Caterina et al. (1997). The capsaicin receptor: a heat-activated ion channel in the pain pathway. Nature 389, 816-824.
10. Chou et al. (2004). Resiniferatoxin binds to the capsaicin receptor (TRPV1) near the extracellular side of the S4 transmembrane domain. Biochemistry 43, 2501-2511.
11. Chuang et al. (2001). Bradykinin and nerve growth factor release the capsaicin receptor from Ptdlns (4,5)P2-mediated inhibition. Nature 411, 957-962.
12. Chung et al. (1990). Characterization of single channel currents using digital signal processing techniques based on Hidden Markov Models. Philos Trans R Soc Lond B Biol Sci 329, 265-285.
13. Cruz et al. (1997). Desensitization of bladder sensory fibers by intravesical capsaicin has long lasting clinical and urodynamic effects in patients with hyperactive or hypersensitive bladder dysfunction. J Urol 157, 585-589.
14. Davis et al. (2000). Vanilloid receptor-1 is essential for inflammatory thermal hyperalgesia. Nature 405, 183-187.
15. De Petrocellis et al. (2001). The vanilloid receptor (TRPV1) mediated effects of anandamide are potently enhanced by the cAMP-dependent protein kinase. J Neurochem 77, 1660-1663.
16. Dinh et al. (2004). Substance P expression in TRPV1 and trkA-positive dorsal root ganglion neurons innervating the mouse lung. Respir Physiol Neurobiol 144, 15-24.
17. Dinis et al. (2004). Anandamide-evoked activation of vanilloid receptor 1 contributes to the development of bladder hyperreflexia and nociceptive transmission to spinal dorsal horn neurons in cystitis. JNeurosci 24, 11253-11263.
18. Docherty et al. (1996). Inhibition of calcineurin inhibits the desensitization of capsaicin-evoked currents in cultured dorsal root ganglion neurons from adult rats. Pflugers Arch 431, 828-837.
19. Doyle et al. (2002). Vanilloid receptors presynaptically modulate cranial visceral afferent synaptic transmission in nucleus tractus solitarius. J Neurosci 22, 8222-8229.
20. Gavva et al. (2004). Molecular determinants of vanilloid sensitivity in TRPV1. J Biol Chem 279, 20283-20295.
21. Giannantoni et al. (2004). Intravesical resiniferatoxin versus botulinum-A toxin injections for neurogenic detrusor overactivity: a prospective randomized study. J Urol 172, 240-243.
22. Harvey et al. (1995). Activation of protein kinase C by the capsaicin analogue resiniferatoxin in sensory neurones. J Neurochem 65, 1309-1317.
23. Helyes et al. (2004). Antiinflammatory and analgesic effects of somatostatin released from capsaicin-sensitive sensory nerve terminals in a Freund's adjuvant-induced chronic arthritis model in the rat. Arthritis Rheum 50, 1677-1685.
24. Huang et al. (2002). An endogenous capsaicin-like substance with high potency at recombinant and native vanilloid TRPV1 receptors. Proc Natl Acad Sci USA 99, 8400-8405.
25. Hwang et al. (2000). Direct activation of capsaicin receptors by products of lipoxygenases: endogenous capsaicin-like substances. Proc Natl Acad Sci USA 97, 6155-6160.
26. lida et al. (2003). TRPV1 activation and induction of nociceptive response by a non-pungent capsaicin-like compound, capsiate. Neuropharmacology 44, 958-967.
27. Julius et al. (2001). Molecular mechanisms of nociception. Nature 413, 203-210.
28. Jung et al. (1999). Capsaicin binds to the intracellular domain of the capsaicin-activated ion channel. J Neurosci 19, 529-538.
29. Jung et al. (2002). Agonist recognition sites in the cytosolic tails of vanilloid receptor 1. J Biol Chem 277, 44448-44454.
30. Karai et al. (2004). Deletion of vanilloid receptor 1-expressing primary afferent neurons for pain control. J Clin Invest 113, 1344-1352.
31. Kim et al. (2003). Intravesical resiniferatoxin for refractory detrusor hyperreflexia: a multicenter, blinded, randomized, placebo-controlled trial. J Spinal Cord Med 26, 358-363.
32. Koplas et al. (1997). The role of calcium in the desensitization of capsaicin responses in rat dorsal root ganglion neurons. J Neurosci 17, 3525-3537.
33. Lazzeri et al. (1998). Urodynamic effects of intravesical resiniferatoxin in humans: preliminary results in stable and unstable detrusor. J Urol 158, 2093-2096.
34. Lazzeri et al. (2004a). Intravesical infusion of resiniferatoxin by a temporary in situ drug delivery system to treat interstitial cystitis: a pilot study. Eur J Urol 45, 98-102.
35. Lazzeri et al. (2004b). Intravesical vanilloids and neurogenic incontinence: ten years experience. Urol Int 72, 145-149.
36. Lazzeri et al. (2004c). Immunohistochemical evidence of vanilloid receptor 1 in normal human urinary bladder. Eur Urol 46, 792-798.
37. Linard et al. (2003). Involvement of primary afferent nerves after abdominal irradiation: consequences on ileal contractile activity and inflammatory mediator release in the rat. Dig Dis Sci 48, 688-697.
38. Lundberg et al. (1983). Substance P and capsaicin-induced contraction of human bronchi. Acta Physiol Scand 119, 49-53.
39. Marinelli et al. (2003). Presynaptic facilitation of glutamatergic synapses to dopaminergic neurons of the rat substantia nigra by endogenous stimulation of vanilloid receptors. J Neurosci 23, 3136-3144.
40. Marinelli et al. (2002). Capsaicin activation of glutamatergic synaptic transmission in the rat locus coeruleus in vitro. J Physiol 543, 531-540.
41. Marshall et al. (2003). Activation of vanilloid receptor 1 by resiniferatoxin mobilizes calcium from inositol 1,4,5trisphosphate-sensitive stores. Br J Pharmacol 138, 172-176.
42. Medical News Today, Aug. 25, 2005 available at http://www.medicalnewstoday.com/medicalnews.php?newsid=29632 (as of Jun. 7, 2006).

43. Mitchell et al. (1997). Role of nitric oxide in the dilator actions of capsaicin-sensitive nerves in the rabbit coronary circulation. Neuropeptides 31, 333-338.
44. Nakatsuka et al. (2002). Activation of central terminal vanilloid receptor-1 receptors and alpha beta-methylene-ATP-sensitive P2X receptors reveals a converged synaptic activity onto the deep dorsal horn neurons of the spinal cord. J Neurosci 22, 1228-1237.
45. National Institute of Dental and Craniofacial Research (NIDCR), May 3, 2004, Animal Studies Show Promise Treating Severe Chronic Pain, available at http://www.nih.gov/news/pr/may2004/nidcr-03.htm (as of Jun. 6, 2006).
46. Neubert et al. (2003). Peripherally induced resiniferatoxin analgesia. Pain 104, 219-228.
47. Oroszi et al. (1999). Interplay between nitric oxide and CGRP by capsaicin in isolated guinea-pig heart. Pharmacol Res 40, 125-128.
48. Pain Management Health Center, at http://www.webmd.com/diseases_and_conditions/pain.htm (as of Jun. 5, 2006).
49. Palma et al. (2004). Resiniferatoxin for detrusor instability refractory to anticholinergics. Int Braz J Urol 30, 53-58.
50. Payne et al. (2005). Intravesical resiniferatoxin for the treatment of interstitial cystitis: a randomized, double-blind, placebo controlled trial. J Urol 173, 1590-1594.
51. Premkumar et al. (2002). Single-channel properties of native and cloned rat vanilloid receptors. J Physiol 545, 107-117.
52. Premkumar et al. (2004). Enhancement of potency and efficacy of NADA by PKC-mediated phosphorylation of vanilloid receptor. J Neurophysiol 91, 1442-1449.
53. Premkumar et al. (1997). Subconductance states of a mutant NMDA receptor channel kinetics, calcium, and voltage dependence. J Gen Physiol 109, 181-189.
54. Qin et al. (1996). Estimating single-channel kinetic parameters from idealized patch-clamp data containing missed events. Biophys J 70, 264-280.
55. Raisinghani et al. (2005). Activation of transient receptor potential vanilloid 1 (TRPV1) by resiniferatoxin. J Physiol 567.3, 771-786.
56. Roberts et al. (2004). [3H]Resiniferatoxin autoradiography in the CNS of wild-type and TRPV1 null mice defines TRPV1 (TRPV1-1) protein distribution. Brain Res 995, 176-183.
57. Statement by National Institutes of Health (NIH), U.S. Dept. of Health and Human Services, on An Update of NIH Pain Research and Related Program Initiatives, Dec. 8, 2005, available at http://www.theacpa.org/cp_{—}02.asp (as of Jun. 5, 2006).
58. Szabo et al. (2002). Pharmacological characterization of vanilloid receptor located in the brain. Brain Res Mol Brain Res 98, 51-57.
59. Szabo et al. (1999). Epidural resiniferatoxin induced prolonged regional analgesia to pain. Brain Res 840, 92-98.
60. Szallasi et al. (1989). Resiniferatoxin, a phorbolrelated diterpene, acts as an ultrapotent analog of capsaicin, the irritant constituent in red pepper. Neuroscience 30, 515-520.
61. Szallasi et al. (1990a). Resiniferatoxin and its analogs provide novel insights into the pharmacology of the vanilloid (capsaicin) receptor. Life Sci 47, 1399-1408.
62. Szallasi et al. (1990b). Specific binding of resiniferatoxin, an ultrapotent capsaicin analog, by dorsal root ganglion membranes. Brain Res 524, 106-111.
63. Szallasi et al. (1996). Vanilloid receptors: new insights enhance potential as a therapeutic target. Pain 68, 195-208.
64. Tominaga et al. (1998). The cloned capsaicin receptor integrates multiple pain-producing stimuli. Neuron 21, 531-543.
65. VanBuren et al. (2005). Sensitization and translocation of TRPV1 by insulin and IGF-1. Mol Pain 1, 17.
66. Vass et al. (2004). Co-localization of the vanilloid capsaicin receptor and substance P in sensory nerve fibers innervating cochlear and vertebro-basilar arteries. Neuroscience 124, 919-927.
67. Voets et al. (2004). The principle of temperature-dependent gating in cold- and heat-sensitive TRP channels. Nature 430, 748-754.
68. Vyklicky et al. (2003). Vanilloid receptor TRPV1 is not activated by vanilloids applied intracellularly. Neuroreport 14, 1061-1065.
69. Walpole et al. (1996). Similarities and differences in the structure-activity relationships of capsaicin and resiniferatoxin analogues. J Med Chem 39, 2939-2952.
70. Zheng et al. (2003). Vanilloid receptors in hearing: altered cochlear sensitivity by vanilloids and expression of TRPV1 in the organ of corti. J Neurophysiol 90, 444-455.
71. Zygmunt et al. (1999). Vanilloid receptors on sensory nerves mediate the vasodilator action of anandamide. Nature 400, 452-457.

What is claimed is:

1. A method of inhibiting nociceptive transmission by TRPV1-containing neurons comprising:
contacting the TRPV1-containing neurons with an effective amount of a TRPV1 agonist to induce at least one of nerve terminal depolarization block and nerve terminal death, wherein the effective amount does not result in significant permanent damage to cell bodies in the TRPV1-containing neurons.

2. The method of claim 1 wherein the TRPV1 agonist is selected from the group consisting of resiniferatoxin and tinyatoxin.

3. The method of claim 2 wherein the TRPV1 agonist is resiniferatoxin.

4. The method of claim 3 wherein the resiniferatoxin is administered intrathecally.

5. The method of claim 4 wherein the effective amount is about 0.01 to about 5 micrograms/kilogram of resiniferatoxin.

6. The method of claim 5 wherein the resiniferatoxin is provided in a volume of about 0.01 to about 0.1 milliliters of a pharmaceutically acceptable carrier.

7. The method of claim 3 wherein the resiniferatoxin is administered intraarticularly.

8. The method of claim 7 wherein the effective amount is about 0.01 to about 5 micrograms/kilogram of resiniferatoxin.

9. The method of claim 7 wherein the resiniferatoxin is provided in a volume of about 0.01 to about 0.1 milliliters of a pharmaceutically acceptable carrier.

10. The method of claim 3 wherein the resiniferatoxin is administered topically.

11. The method of claim 10 wherein the resiniferatoxin is administered using a solution of resiniferatoxin at about 5 to about 50 nM in a pharmaceutically acceptable carrier.

12. A method of treating an inflammatory pain condition comprising:
administering an effective amount of a TRPV1 agonist to a patient to induce at least one of nerve terminal depolarization block and nerve terminal death in TRPV1-containing neurons, wherein the effective amount does not result in significant permanent damage to cell bodies in TRPV1-containing neurons.

13. The method of claim 12 wherein the TRPV1 agonist is selected from the group consisting of resiniferatoxin and tinyatoxin.

14. The method of claim 13 wherein the TRPV1 agonist is resiniferatoxin.

15. The method of claim 14 wherein the resiniferatoxin is administered intrathecally.

16. The method of claim 15 wherein the effective amount is about 0.01 to about 5 micrograms/kilogram of resiniferatoxin.

17. The method of claim 16 wherein the effective amount is provided in a volume of about 0.01 to about 0.1 milliliters of a pharmaceutically acceptable carrier.

18. The method of claim 14 wherein the resiniferatoxin is administered intraarticularly.

19. The method of claim 18 wherein effective amount is about 0.01 to about 5 micrograms/kilogram of resiniferatoxin.

20. The method of claim 19 wherein the effective amount is provided in a volume of about 0.01 to about 0.1 milliliters of a pharmaceutically acceptable carrier.

21. The method of claim 14 wherein the resiniferatoxin is administered topically.

22. The method of claim 21 wherein the resiniferatoxin is administered using a solution of resiniferatoxin at about 5 to about 50 nM in a pharmaceutically acceptable carrier.

23. A method of treating an inflammatory pain condition comprising:
administering an effective amount of a TRPV1 agonist to a patient to induce at least one of nerve terminal depolarization block and nerve terminal death in TRPV1-containing neurons.
